(19) United States
(12) Patent Application Publication
(10) Pub. No.: US 2023/0190121 A1
(43) Pub. Date: Jun. 22, 2023

(54) MEASUREMENT DEVICE AND ESTIMATION SYSTEM

(71) Applicant: KYOCERA CORPORATION, Kyoto-shi, Kyoto (JP)

(72) Inventor: Takahiro WATANABE, Yokohama-shi, Kanagawa (JP)

(21) Appl. No.: 17/925,517

(22) PCT Filed: May 26, 2021
(86) PCT No.: PCT/JP2021/019904

§ 371 Date: Nov. 15, 2022

(30) Foreign Application Priority Data
May 27, 2020 (JP) 2020-092487

(51) Int. Cl. A61B 5/024; G01F 1/661; A61B 5/11; A61B 5/00; A61B 5/026

(52) U.S. Cl. CPC A61B 5/02416; G01F 1/661; A61B 5/02438; A61B 5/11; A61B 5/6817; A61B 5/4812; A61B 5/0261

(57) ABSTRACT

A measurement device includes a light emitting element configured to irradiate a blood vessel of a subject with light, a light receiving element configured to output an optical signal from the subject as an electric signal, and a controller electrically connected to the light receiving element. The controller estimates a heart rate of the subject on the basis of a part of a plurality of frequency components included in output of the light receiving element.

### TECHNICAL FIELD

The present disclosure relates to a measurement device and an estimation system.

### BACKGROUND OF INVENTION

In the related art, a known method is used to measure the degree of motion of a measurement object in a fluid by receiving scattered light from the measurement object. For example, a fluid evaluation device disclosed in Patent Document 1 receives scattered light from a measurement object, and outputs a flow rate or a flow speed of a fluid on the basis of the relationship between light reception amount information included in a light reception signal and information based on a beat signal caused by a light Doppler shift.

### CITATION LIST

### Patent Literature

Patent Document 1: JP 2017-113320 A (published on Jun. 29, 2017)

### SUMMARY

A measurement device according to one aspect of the present disclosure includes: a light emitting element configured to irradiate a blood vessel of a subject with light; a light receiving element configured to output an optical signal from the subject as an electric signal; and a controller electrically connected to the light receiving element, in which the controller estimates a heart rate of the subject on the basis of a part of a plurality of frequency components included in output of the light receiving element.

A measurement device according to one aspect of the present disclosure includes: a signal generator configured to generate a light reception signal by receiving scattered light from a blood flow of a subject; a detector configured to detect a change in an orientation of the subject; and a pattern data generator configured to generate pattern data indicating a fluctuation pattern of a blood flow rate of the subject by analyzing the light reception signal corresponding to a detection result of the detector.

An estimation system according to one aspect of the present disclosure includes: a measurement device; and a computing device including a second controller configured to communicate with the measurement device, in which the second controller includes a third estimation unit estimating a sleep stage of the subject on the basis of the heart rate estimated by the measurement device.

### BRIEF DESCRIPTION OF THE DRAWINGS

FIG. 1 is a block diagram illustrating an example of a configuration of a measurement device according to one embodiment.

FIG. 2 is an external view illustrating an example of the measurement device worn in an ear of a subject.

FIG. 3 is an example illustrating generated pattern data.

FIG. 4 is a graph comparing data indicating a heart rate estimated by the measurement device and heart rate data measured using polysomnography (PSG).

FIG. 5 is a flowchart for explaining a process performed by the measurement device according to one aspect of the present disclosure.

FIG. 6 is a block diagram illustrating a configuration of a measurement device that performs calibration on the basis of changes in the orientation of a subject.

FIG. 7 is a block diagram illustrating a configuration of a measurement device according to another embodiment.

FIG. 8 is a block diagram illustrating a configuration of an estimation system according to another embodiment.

### DESCRIPTION OF EMBODIMENTS

### First Embodiment

One aspect of the present disclosure will be described in detail below. A known measurement device in the related art is required to measure a heart rate on the basis of the blood flow of a subject. On the other hand, according to one aspect of the present disclosure, the heart rate can be measured more accurately on the basis of the blood flow of the subject.

A measurement device 1 according to one embodiment of the present disclosure is an example of a measurement device (laser Doppler flow rate measurement device) using laser Doppler flowmetry (LDF). The measurement device 1 according to one aspect of the present disclosure generates pattern data indicating a flow rate of a fluid and a fluctuation pattern of the flow rate by using the LDF. The measurement device 1 may be a device for irradiating a fluid (for example, a blood flow) inside a subject with a laser beam and estimates a heart rate of the subject on the basis of an optical signal from a moving object (for example, a blood cell) and a stationary object (for example, a blood vessel) included in the fluid. As an example, the optical signal may include scattered light. The measurement device 1 may generate pattern data indicating a fluctuation pattern of biological information of the subject on the basis of scattered light, and estimate the heart rate of the subject on the basis of the pattern data.

The subject may be any organism serving as a measurement target. That is, the subject is not limited to a person, and may be an animal such as a dog or a cat. The biological information generated by the measurement device 1 is not limited to the blood flow rate of the subject, and may be, for example, a flow speed of a blood flow. The measurement target is not limited to the blood flow of the subject, and is a fluid that generates scattered light as a laser beam irradiation result. Hereinafter, in the present specification, the description of “A to B” for two numbers A and B is referred to as “A or more and B or less” unless otherwise specified.

### Principle of LDF

When a fluid is irradiated with a laser beam, the emitted laser beam is scattered by (i) moving objects included in the fluid and moving together with the fluid and (ii) stationary objects such as pipes through which the fluid flows, so that scattered light is generated. In general, the moving objects cause a non-uniform complex refractive index in the fluid.

The scattered light generated by the moving objects moving together with the fluid undergoes a wavelength shift due to a Doppler effect corresponding to the flow speed of the moving objects. On the other hand, the scattered light generated by the stationary objects undergoes no wavelength shift. Since these scattered lights cause optical interference, an optical beat is observed.

In LDF, a value corresponding to the flow rate of the fluid can be calculated by analyzing a frequency intensity distribution (frequency power spectrum) of the optical signal including the optical beat.

### Configuration of Measurement Device 1

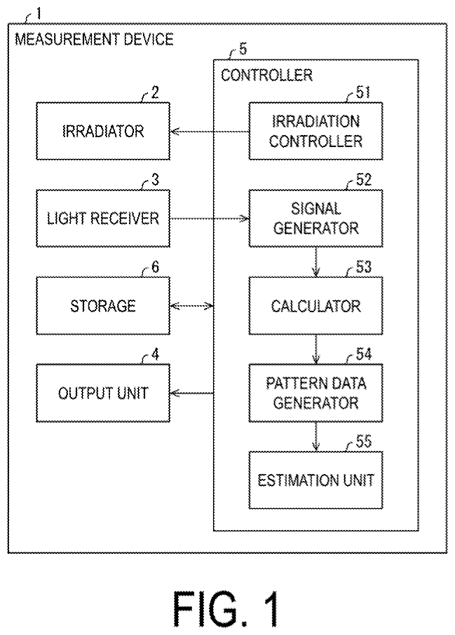

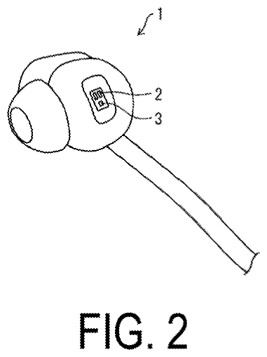

FIG. 1 is a block diagram illustrating an example of a configuration of the measurement device 1 according to one embodiment. FIG. 2 is an external view illustrating an example of the measurement device 1 worn in an ear of a subject. As illustrated in FIG. 1, the measurement device 1 includes an irradiator 2 (light emitting element), a light receiver 3 (light receiving element), an output unit 4, and a controller 5.

The shape of the measurement device 1 is not particularly limited as long as the light receiver 3 can receive an optical signal from the subject. The measurement device 1 may be a wearable device worn on a body of the subject. The measurement device 1 may be worn at a position (for example, hand, finger, trunk, leg, neck, and the like) where the light receiver 3 can receive the optical signal from the subject. The measurement device 1 according to the present embodiment can be worn in an ear of the subject. That is, the measurement device 1 according to the present embodiment can measure biological information on blood flow in the ear. In general, the ear moves less than a finger or the like. Therefore, when the measurement device 1 is worn in an ear, noise caused by the movement of the human body can be reduced compared to when the measurement device 1 is worn on a finger or the like. FIG. 2 illustrates an example of the external appearance of the measurement device having a shape that can be worn in an ear, particularly an ear hole, of the subject.

The irradiator 2 is a light emitting element capable of irradiating a fluid with light having a desired wavelength and intensity under the control of an irradiation controller 51. The irradiator 2 may be a laser diode capable of emitting a laser beam. The laser beam emitted from the irradiator 2 may have a wavelength of, for example, 700 nm to 900 nm. The light emitted to the subject by the irradiator 2 is scattered by blood cells moving together with the blood flow and blood vessels through which blood flows, thereby generating scattered light.

The light receiver 3 is a light receiving element capable of receiving scattered light (optical signal) generated as a result of irradiating the subject with the laser beam and outputting an electric signal corresponding to the scattered light. The light receiver 3 may be a photodiode that generates an electric signal having an intensity corresponding to the received light. The light receiver 3 outputs the generated electric signal to a signal generator 52. The light receiver 3 may generate a light reception signal each time the scattered light is received.

In the case of the measurement device 1 having a shape that can be worn in an ear hole of the subject, as illustrated in FIG. 2, the irradiator 2 may be disposed at a position where the laser beam can be emitted toward an auricle of the subject (that is, toward capillaries of the auricle). The light receiver 3 may also be disposed at a position where scattered light can be received from the capillaries receiving the laser beam.

The output unit 4 acquires various data related to the blood flow of the subject and generated in the controller 5, and outputs the acquired data to an external device. As an example, the output unit 4 acquires pattern data indicating a pattern of the blood flow rate of the subject from a pattern data generator 54, and outputs the acquired pattern data to an external device (not illustrated). The output unit 4 may acquire data indicating a heart rate of the subject estimated by, for example, an estimation unit 55 to be described below, and output the acquired data to the external device.

The external device may be any device that acquires various data generated by the measurement device 1. The external device may be a device that performs additional computations by using various data generated by the measurement device 1, or may be a display device that displays various data acquired from the measurement device 1. Alternatively, the external device may be a storage device such as a universal serial bus (USB) memory that stores various data generated by the measurement device 1. As an example, when the measurement device 1 is connected to an external computing device, the output unit 4 may be a communication module capable of transmitting various data to the computing device in wired or wireless communication. When the measurement device 1 is a device for displaying various data, the output unit 4 may be a display such as a liquid crystal display or may display various data acquired from the controller 5.

The storage 6 is a storage region where various data used in the measurement device 1 are stored. For example, the storage 6 may store a first predetermined value and a second predetermined value used in the pattern data generator 54. Specific examples of the first predetermined value and the second predetermined value will be described below.

The controller 5 is electrically connected to the light receiver 3. As illustrated in FIG. 1, the controller 5 includes the irradiation controller 51, the signal generator 52, a calculator 53, the pattern data generator 54, and the estimation unit 55. The controller 5 estimates the heart rate of the subject on the basis of output having a part of a plurality of frequency components included in the output of the light receiver 3. The irradiation controller 51 controls the irradiator 2 to emit a laser beam having a desired wavelength.

The signal generator 52 acquires an electric signal corresponding to the intensity of the scattered light from the light receiver 3. The signal generator 52 may perform an A/D conversion process on the electric signal that is the output from the light receiver 3, and generate a light reception signal corresponding to the intensity of the scattered light. The signal generator 52 outputs the generated light reception signal to the calculator 53.

The calculator 53 acquires the light reception signal from the signal generator 52. The calculator 53 analyzes the acquired light reception signal and calculates frequency analysis data indicating a signal intensity for each frequency of the light reception signal. As an example, the calculator 53 may analyze the acquired light reception signal by using a method such as fast Fourier transformation (FFT). The frequency analysis data calculated by the calculator 53 may include data indicating a signal intensity in a frequency band of 1 kHz to 20 kHz, for example. The calculator 53 may perform the analysis each time the light reception signal is acquired. The calculator 53 may output the calculated frequency analysis data to the pattern data generator 54.

The pattern data generator 54 acquires the frequency analysis data from the calculator 53, and generates pattern data indicating a fluctuation pattern of the blood flow rate of the subject on the basis of the frequency analysis data. As an example, the pattern data generator 54 may calculate a primary moment sum X of the acquired frequency analysis data. More specifically, the pattern data generator 54 may calculate the primary moment sum X of the acquired frequency analysis data by using the following equation. When the frequency analysis data includes the data indicating a signal intensity in the frequency band of 1 kHz to 20 kHz, the pattern data generator 54 calculates the primary moment sum X in the frequency band of 1 kHz to 20 kHz by using the following equation.

X=Σfx×P(fx)

In the formula above, fx denotes a frequency and P(fx) denotes the value of a signal intensity at the frequency fx.

The primary moment sum calculated by the pattern data generator 54 on the basis of the frequency analysis data may be a value proportional to the blood flow rate of the subject. The pattern data generator 54 may generate pattern data indicating a fluctuation pattern of the blood flow rate of the subject over time by calculating the primary moment sum for each of a plurality of frequency analysis data. The pattern data generator 54 may also generate the pattern data by using data included in a part of the frequency band among the data included in the frequency analysis data. The pattern data generator 54 outputs the generated pattern data to the output unit 4 and the estimation unit 55.

The pattern data generator 54 may also perform calibration for changing the frequency band used for generating the pattern data. As an example, the pattern data generator 54 may change the frequency band used for generating the pattern data during measurement according to the form of the generated pattern data. The criteria for determining the form of the pattern data are not particularly limited. For example, the pattern data generator 54 may change a frequency band to be used on the basis of the shape of the pattern data (waveform shape).

The pattern data generator 54 may also generate the pattern data by using a part of the entire frequency band included in the frequency analysis data. Specifically, the pattern data generator 54 may acquire data indicating the heart rate of the subject from the estimation unit 55.

The pattern data generator 54 may compare an average value of the heart rate of the subject within a certain period (for example, 30 seconds) with the first predetermined value set in advance, and change the frequency band used for generating the pattern data according to the comparison result. When the average value of the heart rate of the subject is a value that differs from the first predetermined value by 20 beats per minute (bpm) or more, the pattern data generator 54 may determine that the generated pattern data is inaccurate data and change the frequency band used for generating the pattern data. When the measurement device 1 is a device for measuring the heart rate of a person (subject), the first predetermined value is the average value of the heart rate of a normal person within a certain period during rest, for example, 60 bpm.

For example, when the average heart rate of each subject within a certain period during rest is acquired in advance by using an electrocardiograph or the like and is stored in the storage 6, a predetermined value to be compared with the average value of the heart rate of the subject may be set for each subject. The predetermined value set for each subject and compared with the average value of the heart rate of the subject is referred to as the second predetermined value. The second predetermined value may be, for example, a pre-acquired average heart rate of each subject within a certain period during rest. When the measurement device 1 performs measurement for the subject, the pattern data generator 54 acquires the second predetermined value of the subject by referring to the storage 6.

The pattern data generator 54 may compare the second predetermined value of a subject A with an average value of the heart rate of the subject A acquired from the estimation unit 55. As an example, when the average value of the heart rate of the subject A acquired from the estimation unit 55 is a value that differs from a reference heart rate of the subject A by 10 bpm or more, the pattern data generator 54 may determine that the generated pattern data is inaccurate data. By setting the second predetermined value for each subject, the pattern data generator 54 can more accurately determine whether the pattern data is appropriate. The above value (for example, 10 bpm or 20 bpm) of the reference for the difference between the first predetermined value/the second predetermined value and the average heart rate of the subject is an example, and is not limited thereto.

When the generated pattern data is determined to be inaccurate data, the pattern data generator 54 generates pattern data again. In this case, when calculating the primary moment sum, the pattern data generator 54 calculates the primary moment sum by using a part of the entire frequency band included in the acquired frequency analysis data. The part of the frequency band used by the pattern data generator 54 is a band that is less affected (by noise) due to components other than blood cells.

Low-frequency band data is likely to include noise caused by scattered light scattered by substances other than blood cells. Therefore, when the frequency analysis data includes data in the band of 1 kHz to 20 kHz, the pattern data generator 54 may perform the calculation by using data in the band of 8 kHz to 20 kHz as an example. The frequency band used for the calculation by the pattern data generator 54 may be preset for each subject.

The method for selecting a part of the frequency band used for computation by the pattern data generator 54 is not limited to the above. For example, the pattern data generator 54 may perform a known method such as sine curve fitting on the frequency analysis data, and select a band to be used for generating pattern data on the basis of the magnitude of deviation from a sine curve. Specifically, the shape of frequency analysis data serving as a reference may be set in advance, and a frequency band indicating a value that differs from the reference by a certain threshold value or more may be regarded as noise. The pattern data generator 54 may use data at frequencies, other than the frequency band regarded as noise, for computation. The above-described threshold value is not particularly limited, and may be appropriately set according to the accuracy of pattern data to be acquired.

The pattern data generator 54 may also repeat changing the band used for generating the pattern data until the pattern data has an accurate waveform. The fact that the pattern data has an accurate waveform means that the result of comparing the average of the heart rate of the subject within a certain period with a predetermined value satisfies a predetermined condition to be described below. Thus, the measurement device 1 can generate more accurate pattern data, and accurately and stably acquire data such as the blood flow rate and heart rate of the subject.

The estimation unit 55 acquires the pattern data from the pattern data generator 54. The estimation unit 55 may refer to the waveform of the acquired pattern data and measure the number of peaks included in the waveform, thereby estimating the average heart rate of the subject. The estimation unit 55 may also acquire the average value of the number of peaks included in the waveform within a certain period, and output data indicating the average value of the heart rate of the subject to the pattern data generator 54.

In the related art, the intensity of the light reception signal varies, which has made it difficult to accurately generate pattern data indicating a fluctuation pattern of a blood flow rate of a subject. The inventors of the present disclosure have found that, when the frequency band used for generating the pattern data is appropriately adjusted, for example, even though the intensity of the light reception signal is weak, the pattern data can be generated accurately.

The pattern data generator 54 may also change a frequency band in the frequency analysis data used for generating the pattern data, on the basis of the result of the calibration. That is, the pattern data generator 54 generates the pattern data by using frequency analysis data in a band other than a frequency band including data that is noisy or does not allow an accurate heart rate value to be estimated. Thus, the measurement device 1 can stably generate highly accurate pattern data even when the intensity of the light reception signal varies.

The capillaries of an ear have a lower density of capillaries and a lower blood flow rate than sites such as fingertips. Compared with a finger, the device is less tightly worn in an ear hole and the device itself is easily detached. Therefore, in the related art, when a measurement device using LDF is worn in an ear of a subject and pattern data is generated from capillaries of an ear hole, the light reception signal of scattered light tends to be weak, which makes it difficult to acquire accurate data.

On the other hand, the measurement device 1 according to one aspect of the present disclosure performs calibration before measurement, which makes it possible to perform the measurement by using only a frequency band in which accurate pattern data can be generated. Therefore, even when the measurement device 1 is worn in an ear of a subject, it is possible to accurately generate pattern data and measure a heart rate.

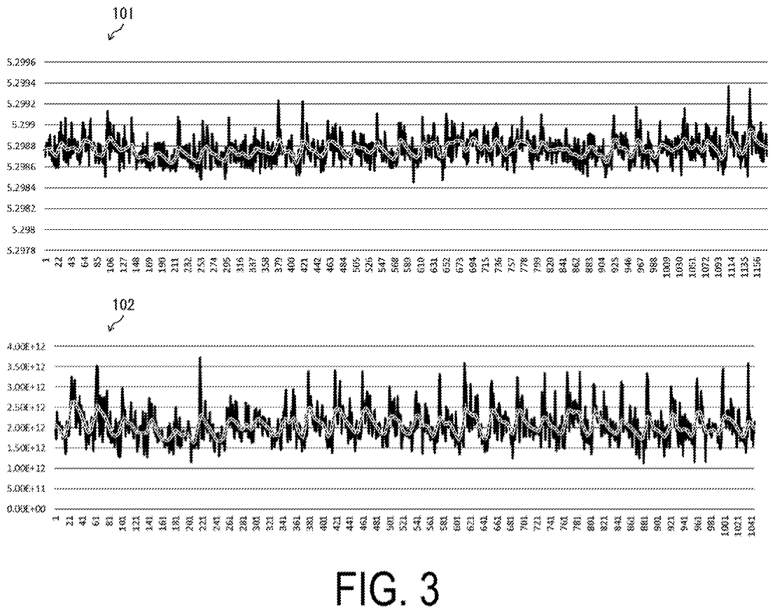

FIG. 3 is an example illustrating generated pattern data. In FIG. 3, a vertical axis indicates a primary moment sum (X) and a horizontal axis indicates time. Note that in the scale of the horizontal axis, scale 1 corresponds to 0.0256 seconds. A graph denoted by reference sign 101 in FIG. 3 indicates pattern data obtained when the measurement device using LDF in the related art is worn in an ear of a subject and performs measurement. A graph denoted by reference sign 102 in FIG. 3 indicates pattern data obtained when measurement is performed similarly using the measurement device 1. As denoted by reference sign 101 in FIG. 3, in the measurement device in the related art, noise is likely to be mixed in a light reception signal and a waveform included in data is distorted. On the other hand, as denoted by reference sign 102 in FIG. 3, the measurement device 1 according to one aspect of the present disclosure generates pattern data by excluding data in a frequency band including noise, which makes it possible to obtain pattern data having a periodic waveform and a clear peak.

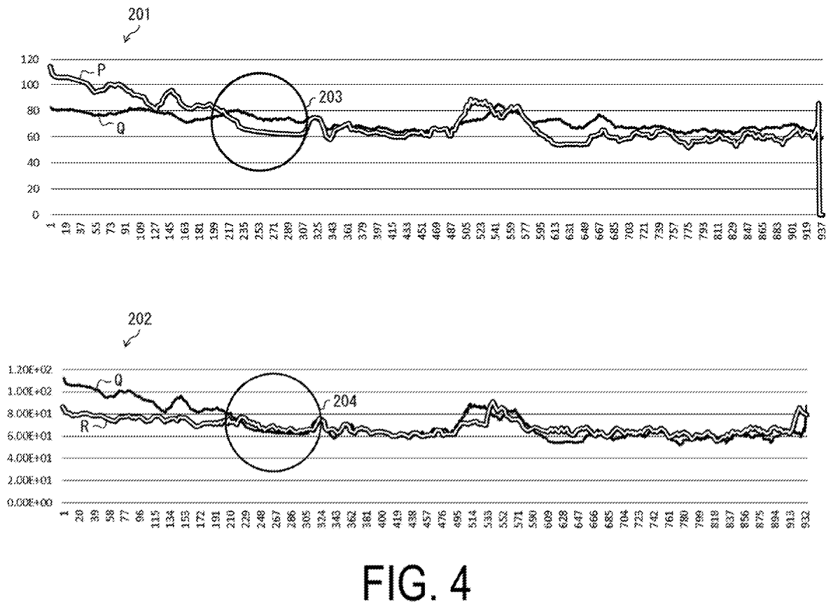

FIG. 4 is a graph comparing data indicating a heart rate estimated by the measurement device 1 with heart rate data measured using polysomnography (PSG). In FIG. 4, the vertical axis indicates heart rate and the horizontal axis indicates time. Note that when the measurement device 1 outputs data indicating the heart rate, the unit of time on the horizontal axis may be appropriately changed according to the interval of the heart rate. The graph denoted by reference sign 201 in FIG. 4 is a graph comparing data indicating the heart rate obtained when the measurement device using LDF in the related art is worn in the ear of the subject and performs measurement with data obtained by measuring the heart rate of the same subject using PSG. The graph denoted by reference sign 202 in FIG. 4 is a graph comparing heart rate data acquired similarly using the measurement device 1 with heart rate data using PSG. As denoted by reference sign 201 in FIG. 4, in the measurement device in the related art, the estimated heart rate deviates from the heart rate measured using PSG, as represented by a portion denoted by reference sign 203. Since the heart rate measured using the PSG can be considered as an accurate value, when the measurement device in the related art is worn in the ear of the subject and used to estimate the heart rate of the subject by using the measurement device, it can be seen that the heart rate is an inaccurate value.

On the other hand, by referring to the graph denoted by reference sign 202 in FIG. 4, when the measurement device 1 is worn in the ear of the subject and is used to estimate the heart rate of the subject by using the measurement device, the deviation between data of the PSG and data by the measurement device 1 in a portion denoted by reference sign 204 (the same time period as that denoted by reference sign 203) is very small compared with that of the related art. That is, it can be seen that in the measurement device 1, data indicating the estimated heart rate of the subject is more accurate than that in the measurement device in the related art.

### Example of Processing Flow of Measurement Device 1

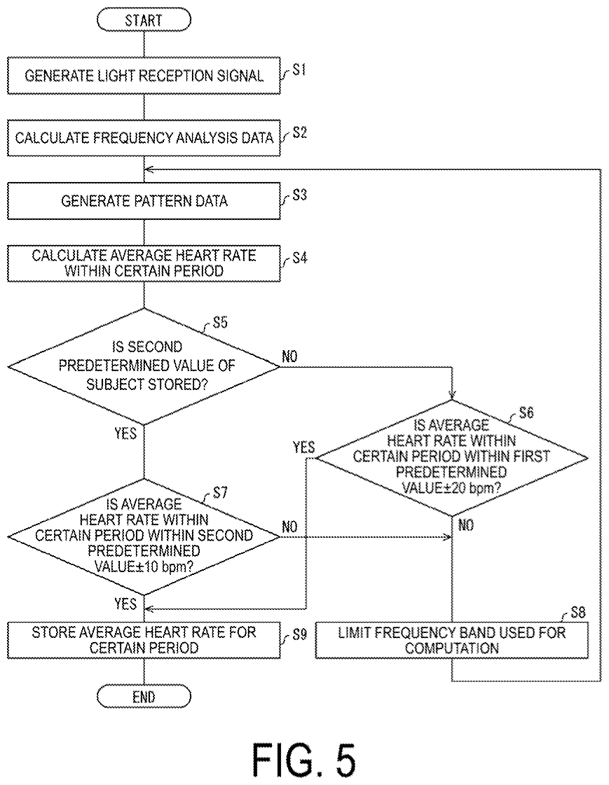

FIG. 5 is a flowchart for explaining a process performed by the measurement device 1 according to one aspect of the present disclosure. An example of the flow of a process (calibration) performed by the measurement device 1 will be described below with reference to FIG. 5. Numerical values used in the following description are examples and are not limited to these numerical values.

First, a laser beam is emitted from the irradiator 2 under the control of the irradiation controller 51. A subject to be measured is irradiated with the laser beam emitted from the irradiator 2. The laser beam emitted to the subject is scattered by the subject. The light receiver 3 receives scattered light generated by the scattering of the laser beam in the subject, and outputs an electric signal corresponding to the intensity of the scattered light to the signal generator 52.

Upon acquiring the electric signal from the light receiver 3, the signal generator 52 performs A/D conversion on the electric signal, and generates a light reception signal corresponding to the intensity of the electric signal, that is, the intensity of the scattered light (step S1). The signal generator 52 outputs the generated light reception signal to the calculator 53.

The calculator 53 analyzes the acquired light reception signal by using FFT, and calculates frequency analysis data indicating the intensity for each frequency of the light reception signal (step S2). The calculator 53 outputs the calculated frequency analysis data to the pattern data generator 54.

The pattern data generator 54 calculates a primary moment sum for the frequency analysis data acquired from the calculator 53, and generates pattern data indicating a fluctuation pattern of a blood flow rate of the subject over time (step S3). The pattern data generator 54 outputs the generated pattern data to the estimation unit 55.

The estimation unit 55 acquires the pattern data and calculates the average value (bpm) of the heart rate of the subject within a certain period (for example, 30 seconds) in the pattern data (step S4). The estimation unit 55 outputs, to the pattern data generator 54, subject heart rate data indicating the calculated average value of the heart rate of the subject.

Upon acquiring the subject heart rate data, the pattern data generator 54 refers to the storage 6, and determines whether the second predetermined value of the subject is stored in the storage 6 (step S5). When the second predetermined value of the subject is not stored in the storage 6 (NO at step S5), the pattern data generator 54 acquires the first predetermined value of the storage 6. The pattern data generator 54 calculates the difference between the acquired first predetermined value and the average heart rate of the subject included in the subject heart rate data, and determines whether the difference is within 20 (step S6).

When the difference between the average heart rate of the subject and the first predetermined value is greater than 20 (NO at step S6), the pattern data generator 54 determines that the pattern data generated at step S3 has an inaccurate waveform. On the other hand, when the difference between the average heart rate of the subject and the first predetermined value is within 20 (YES at step S6), the pattern data generator 54 determines that the pattern data generated at step S3 has an accurate waveform.

At step 5, when the second predetermined value of the subject is stored in the storage 6 (YES at step S5), the pattern data generator 54 acquires the second predetermined value of the subject stored in the storage 6.

Subsequently, the pattern data generator 54 calculates the difference between the average heart rate of the subject and the second predetermined value, and determines whether the difference is within 10 (step S7). When the difference between the second predetermined value and the average heart rate of the subject is greater than 10 (NO at step S7), the pattern data generator 54 determines that the pattern data generated at step S3 has an inaccurate waveform. On the other hand, when the difference between the average heart rate of the subject and the second predetermined value is within 10 (YES at step S7), the pattern data generator 54 determines that the pattern data generated at step S3 has an accurate waveform.

In the case of NO at step S7 or step S8, that is, when the pattern data is determined to have an inaccurate waveform, the pattern data generator 54 limits (changes) a frequency band to be used for computation for generating the pattern data (step S8). In this case, the pattern data generator 54 performs the process of step S3 again by using a part of the frequency band included in the frequency analysis data. The pattern data generator 54 outputs newly generated pattern data to the estimation unit 55, and repeats the same process until the pattern data is determined to have an accurate waveform.

In the case of YES at step S7 or step S8, that is, when the difference between the average heart rate of the subject and the first predetermined value or the second predetermined value is within the reference (20 or 10), the pattern data generator 54 ends the calibration. In other words, when the pattern data is determined to have an accurate waveform, the pattern data generator 54 ends the calibration. The pattern data generator 54 stores a frequency band used for generating pattern data having an accurate waveform in the storage 6 as the second predetermined value of the subject (step S9).

After the calibration is ended, the measurement device 1 starts measurement. The pattern data generator 54 generates pattern data corresponding to a variation in the blood flow rate of the subject by using the frequency band used when the pattern data has an accurate waveform at step S7 or step S8, and outputs the pattern data to the estimation unit 55 and the output unit 4. The estimation unit 55 calculates the heart rate of the subject from the pattern data, and outputs the calculated heart rate to the output unit 4. The output unit 4 outputs data indicating the acquired pattern data and heart rate.

### Variations

In the measurement device 1, the light receiver is able to receive scattered light. Therefore, the measurement device 1 does not necessarily have to include the irradiator and the irradiation controller. In this case, a subject is irradiated with light by an external device that emits the light, and the light receiver receives scattered light generated by the scattering of the light in the subject.

In the embodiment described above, the measurement device 1 performs calibration using the heart rate of the subject as a reference, but the reference used for the calibration is not limited to the heart rate of the subject. For example, the calibration may also be performed on the basis of a detection result of changes in the orientation of the subject.

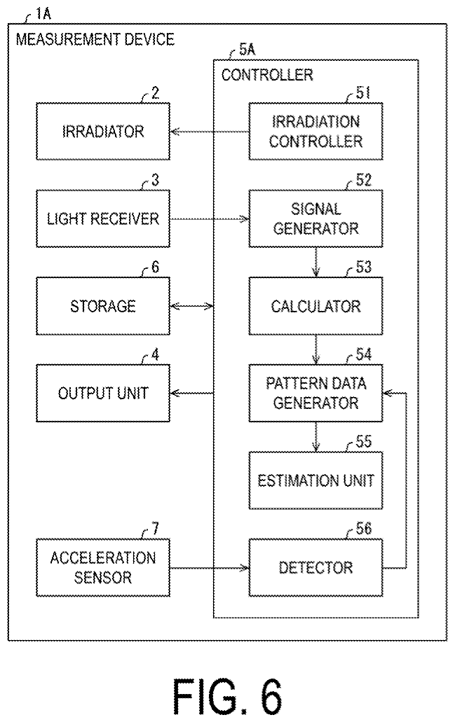

FIG. 6 is a block diagram illustrating a configuration of a measurement device 1A that performs calibration on the basis of changes in the orientation of a subject. As illustrated in FIG. 6, the measurement device 1A includes a controller 5A and an acceleration sensor 7. The controller 5A includes a detector 56. The acceleration sensor 7 is a sensor that outputs an electric signal corresponding to changes in the orientation of the subject. The detector 56 acquires the electric signal from the acceleration sensor 7, and detects the orientation of the subject on the basis of the changes in the intensity of the electric signal.

For example, when the changes in the intensity of the acquired electric signal within a predetermined period are equal to or greater than a certain threshold value, the detector 56 determines that the orientation of the subject has changed. The detector 56 outputs a signal (detection result) indicating the changes in the orientation of the subject to the pattern data generator 54. When the signal is acquired, the pattern data generator 54 limits (changes) a frequency band to be used for generating pattern data, and then generates the pattern data.

When the orientation of the subject has changed, the measurement device 1A is likely to deviate from a state in which the measurement device 1A is first worn by the subject. In such a case, a light reception signal acquired by the measurement device 1A is likely to be unstable. The measurement device 1A can perform calibration according to a result of detection by the acceleration sensor 7 and the detector 56. Accordingly, when the orientation of the subject has changed, the calibration for selecting the frequency for generating the pattern data again can be performed. Thus, the measurement device 1A can generate more stable pattern data and calculate a heart rate.

The pattern data generator 54 may also be configured to perform calibration only when the detector 56 detects changes in the orientation of the subject and the heart rate of the subject after detection of the changes in the orientation is significantly different from the heart rate of the subject before the detection of the changes in the orientation.

The acceleration sensor 7 and the detector 56 need not be devices integrated with the measurement device 1. As an example, the acceleration sensor 7 and the detector 56 are devices communicably connected to the measurement device 1. The acceleration sensor 7 and the detector 56 detect the changes in the orientation of the subject, and transmit information indicating that the orientation of the subject has changed to the measurement device 1 when the changes in the orientation of the subject are detected.

The pattern data generator 54 may also perform calibration on the basis of the intensity of the light reception signal instead of the heart rate of the subject. In this case, the pattern data generator 54 acquires the light reception signal from the signal generator 52. When the intensity of the acquired light reception signal falls lower than a preset threshold value, the pattern data generator 54 limits (changes) a frequency band to be used for generating the pattern data, and then generates the pattern data.

When the measurement device 1 performs no calibration based on the heart rate, the measurement device 1 need not include the estimation unit 55. In this case, the output unit 4 acquires and outputs only the pattern data.

### Second Embodiment

Another embodiment of the present disclosure will be described below. Note that, for convenience of description, a member having the same function as that of a member described in the embodiment described above is denoted by the same reference sign, and description thereof will not be repeated.

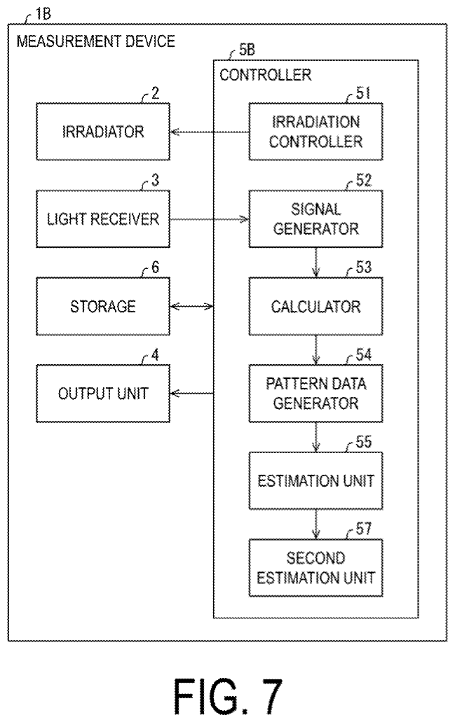

FIG. 7 is a block diagram illustrating a configuration of a measurement device 1B according to another embodiment. The measurement device 1B estimates the state of sleep of a subject (hereinafter, referred to as sleep stage), in addition to the heart rate of the subject. As illustrated in FIG. 7, the measurement device 1B includes a controller 5B including a second estimation unit 57.

The second estimation unit 57 acquires data indicating the heart rate of the subject, and estimates the sleep stage of the subject on the basis of the data. The sleep stage of the subject may be, for example, a stage indicating whether the subject is in an awake state or in a sleep state. The sleep stage may be classified in more detail. For example, the sleep stage may include a state in which the subject is in light sleep such as rapid eye movement (REM) sleep or a state in which the subject is in deep sleep such as non-rapid eye movement (non-REM) sleep. The non-REM sleep may be further classified according to the depth of sleep. For example, the non-REM sleep may be classified into stage 1, stage 2, stage 3, and stage 4 in order of decreasing sleep intensity. A known method is used for estimating the sleep stage from the heart rate of the subject.

### Third Embodiment

Another embodiment of the present disclosure will be described below. Note that, for convenience of description, a member having the same function as that of a member described in the embodiments described above is denoted by the same reference sign, and description thereof will not be repeated.

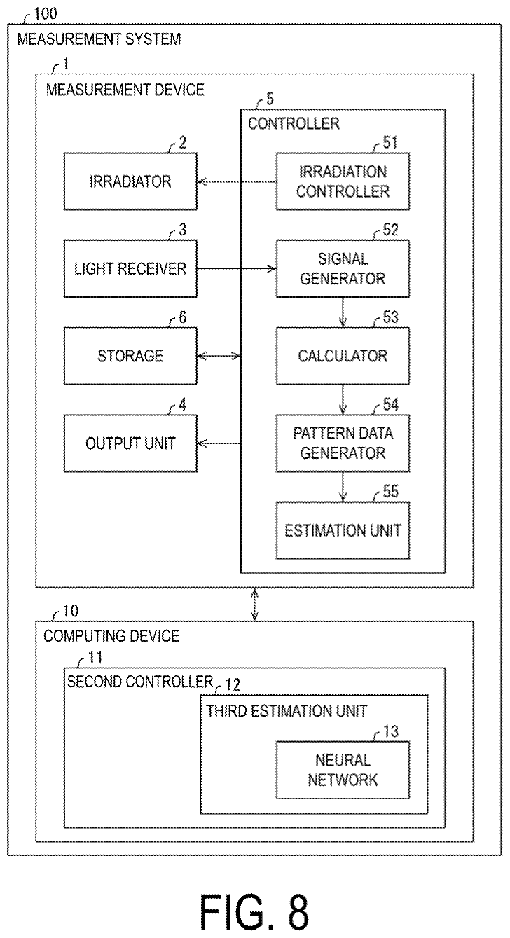

FIG. 8 is a block diagram illustrating a configuration of an estimation system 100 according to another embodiment. As illustrated in FIG. 8, the estimation system 100 according to another embodiment includes the measurement device 1 and a computing device 10. Since the measurement device 1 is the same measurement device as described above, description thereof will be omitted. The estimation system 100 estimates the sleep stage of the subject from the heart rate of the subject estimated by the measurement device 1.

The computing device 10 is a device including a second controller 11 and communicably connected to the measurement device 1. The measurement device 1 transmits data indicating the heart rate of the subject to the computing device 10 via the output unit 4. The second controller 11 controls each component of the computing device 10. As illustrated in FIG. 7, the second controller 11 includes a third estimation unit 12. The third estimation unit 12 acquires the data indicating the heart rate of the subject, and estimates a sleep stage of the subject on the basis of the data. A method for estimating the sleep stage may use the same method as that performed by the second estimation unit 57 in the computing device 10.

The third estimation unit 12 may further include a neural network 13 capable of estimating the sleep stage from the heart rate of the subject. In this case, the neural network 13 may be a neural network pre-learned using the data indicating the heart rate of the subject, as input data to be used for learning, and the sleep stage of the subject when the heart rate has been measured, as teacher data. The neural network 13 acquires the data indicating the heart rate of the subject from the measurement device 1, uses the acquired data as input data, and estimates the sleep stage of the subject from the input data.

The neural network 13 included in the third estimation unit 12 may estimate the sleep stage of the subject by further using data other than the heart rate of the subject. For example, the neural network 13 may acquire pattern data from the measurement device 1 in addition to the heart rate of the subject, and perform estimation using these data. The third estimation unit 12 can improve the accuracy of estimation by increasing the type of input data.

### Example of Software Implementation

Control blocks (particularly, the signal generator 52, the calculator 53, the pattern data generator 54, the estimation unit 55, the detector 56, the second estimation unit 57, and the third estimation unit 12) of the measurement devices 1, 1A, and 1B and the estimation system 100 may be implemented by a logic circuit (hardware) formed in an integrated circuit (IC chip) or the like, or may be implemented by software.

In the latter case, the measurement devices 1, 1A, and 1B and the estimation system 100 include a computer that executes instructions of a program that is software for implementing each function. The computer includes, for example, one or more processors and a computer-readable recording medium that stores the above program. That is, the controllers 5 of the measurement devices 1, 1A, and 1B may be processors. The storage 6 may be a storage medium. Then, in the computer, the processor reads the above program from the recording medium and executes the read program to achieve the object of the present disclosure. As the processor, a central processing unit (CPU) can be used, for example. As the recording medium, a “non-transitory tangible medium”, for example, a tape, a disk, a card, a semiconductor memory, a programmable logic circuit, or the like can be used in addition to a read only memory (ROM). The computer may further include a random access memory (RAM) for loading the above program. The above program may be supplied to the computer via any transmission medium (communication network, broadcast wave, and the like) capable of transmitting the program. One aspect of the present disclosure may be implemented in the form of data signals embedded in a carrier wave in which the above program is embodied by electronic transmission.

The invention according to the present disclosure has been described above based on the drawings and examples. However, the invention according to the present disclosure is not limited to each embodiment described above. That is, the invention according to the present disclosure can be modified in various ways within the scope indicated in the present disclosure, and an embodiment to be obtained by appropriately combining technical means disclosed in different embodiments is also included in the technical scope of the invention according to the present disclosure. In other words, note that a person skilled in the art can easily make various changes or variations based on the present disclosure. Note that these changes or variations are included within the scope of the present disclosure.

### REFERENCE SIGNS

1, 1A, 1B Measurement device
2 Irradiator (light emitting element)
3 Light receiver (light receiving element)
5 Controller
10 Computing device
11 Second controller
12 Third estimation unit
13 Neural network
52 Signal generator
53 Calculator
54 Pattern data generator
55 Estimation unit
56 Detector
57 Second estimation unit
100 Estimation system

1. A measurement device comprising:
a light emitting element configured to irradiate a blood vessel of a subject with light;
a light receiving element configured to output an optical signal from the subject as an electric signal; and
a controller electrically connected to the light receiving element, wherein the controller estimates a heart rate of the subject based on a part of a plurality of frequency components included in the output of the light receiving element.

2. The measurement device according to claim wherein the controller comprises:
a signal generator for generating a light reception signal from the output of the light receiving element.;
a calculator for calculating frequency analysis data indicating a signal intensity for each frequency of the light reception signal;
a pattern data generator for generating pattern data indicating a fluctuation pattern of biological information of the subject based on the frequency analysis data; and
an estimation unit for estimating a heart rate of the subject from the pattern data, and
the pattern data generator generates the pattern data by using a part of an entire frequency band included in the frequency analysis data.

3. The measurement device according to claim 1, wherein the controller comprises:
a signal generator for generating a light reception signal from the output of the light receiving element;
a calculator for calculating frequency analysis data indicating a signal intensity for each frequency of the light reception signal.;
a pattern data generator for generating pattern data indicating a fluctuation pattern of biological information of the subject on the basis of the frequency analysis data; and
an estimation unit for estimating a heart rate of the subject from the pattern data, and
the pattern data generator is able to change a frequency band in the frequency analysis data used for generating the pattern data.

4. The measurement device according to claim 2, wherein
the estimation unit estimates the heart rate of the subject by measuring a number of peaks in the pattern data.

5. The measurement device according to claim 2, wherein
the pattern data generator changes the frequency band used for generating the pattern according to the generated pattern data.

6. The measurement device according to claim 2, further comprising:
a detector for detecting a change in an orientation of the subject, wherein
when the detector detects a change in the orientation of the subject, the pattern data generator changes the frequency band used for generating the pattern data.

7. The measurement device according to claim 2, wherein
the pattern data generator changes the frequency band used for generating the pattern data according to an intensity of the light reception signal.

8. The measurement device according to claim 2, wherein
the pattern data generator changes the frequency band used for generating the pattern data according to a result of comparing an average of a heart rate of the subject within a certain period with a predetermined value.

9. The measurement device according to claim 2, wherein
the pattern data generator repeats changing of the frequency hand used for generating the pattern data until the result of comparing the average of the heart rate of the subject within the certain period with the predetermined value satisfies a predetermined condition.

10. A measurement device comprising:
a signal generator for generating a light reception signal by receiving scattered light from a blood flow of a subject
a detector for detecting a change in an orientation of the subject; and
a pattern data generator for generating pattern data indicating a fluctuation pattern of a blood flow rate of the subject by analyzing the light reception signal corresponding to a detection result of the detector.

11. The measurement device according to claim 1, wherein
the measurement device is worn on a body of the subject.

12. The measurement device according to claim 1, wherein
the measurement device is worn in an ear of the subject.

13. The measurement device according to claim 1, further comprising;
a second estimation unit estimating a sleep stage of the subject on the basis of the heart rate of the subject.

14. An estimation system comprising:
the measurement device according to claim 1; and
a computing device comprising a second controller configured to communicate with the measurement device, wherein
the second controller comprises a third estimation unit estimating a sleep stage of the subject on the based on the heart rate estimated by the measurement device.

15. The estimation system according to claim 14, wherein
the second controller comprises a neural network configured to estimate the sleep stage from the heart rate of the subject.
